(19) United States
(12) Patent Application Publication
(10) Pub. No.: US 2007/0125372 A1
(43) Pub. Date: Jun. 7, 2007

(54) DOSE INHALATION MONITOR

(76) Inventor: Tu-Chuan CHEN, Kaohsiung City (TW)

Correspondence Address: SINORICA, LLC, 528 FALLSGROVE DRIVE, ROCKVILLE, MD, 20850, US

(21) Appl. No.: 11/621,026

(22) Filed: Jan. 8, 2007

(30) Foreign Application Priority Data
Oct. 14, 2005 (TW) 95137829

(51) Int. Cl. A61M 11/00

(52) U.S. Cl. 128/200.23; 128/200.14

(57) ABSTRACT

A dose inhalation monitor includes a main body for containing medicine therein, a dose monitoring unit and a transmission device. The main body has a feeding mouth and a bypass communicated spatially with the feeding mouth. The dose monitoring unit is disposed on the main body, and includes a detector capable of detecting and converting a pressure within the bypass into a voltage level and a processor capable of processing the voltage level in order to generate an oral dose of medicine corresponding to the voltage level. The transmission device is connected to the dose monitoring unit for transmitting the oral dose of medicine to a distal receiver after receipt thereof.

### FIELD OF THE INVENTION

The present invention relates to a medicine feeding apparatus, and more particularly to a dose inhalation monitor.

### BACKGROUND OF THE INVENTION

For those suffering from asthmatic symptoms, rhinitis or bronchitis, an asthmatic tool or a snivel inhaler is generally used for feeding medicine via the mouth. Since asthma and bronchitis in a patient are required to keep under constant observation and the dose thereof is altered depending on the health situation of the patient. The amount of dose being taken and the feeding time interval are required to be recorded for reference during the treating process.

FIG. 1 shows a conventional medicine feeding apparatus 1 presently used in the health circle. The feeding apparatus 1 includes a main body 10 generally of oval-shaped for containing medicine therein. The medicine is usually in powder type. The main body 10 has a lateral side formed with a feeding mouth 12, a dispensing arm 14, a thumb grip 16 and a slidable shield 18. The main body 10 further has a counting meter 19.

The slidable shield 18 is mounted slidably on the lateral side of the main body 10 such that when the feeding apparatus 1 is not in use, the shield 18 covers the feeding mouth 12 and the dispensing arm 14, thereby keeping them hygienic free from pollution. For feeding the medicine, the patient can hook his thumb onto the thumb grip 16 in order to manipulate the dispensing arm 14 by means of his finger 14, thereby producing a dose of medicine from the main body 10. The counting meter 19 will indicate the number of medicine being fed.

Since the dose of medicine is taken by suction, insufficient of the dose can deteriorate the treatment of the patient. Insufficiency in the dose and the inappropriate feeding times can lead to wrong diagnose of the patient condition. Most of the medicine feeding apparatuses presently available in the market are not provided with dose monitoring unit so that the patient must write down the amount of dose, the numbers of feeding time and the time interval between adjacent to feeding operations to tell his doctor upon the next visit or when the doctor conducts the routine checkup. Very often, the patient may forget to write down the amount of dose or the feeding time interval, which, in turn, can lead to wrong diagnose of the patient condition.

Therefore, it is urgently needed to invent a dose inhalation monitor having the ability to record automatically the dose amount, the feeding times and the time of succeeding feeding operations so as to enable a medical practitioner to make a correct diagnose.

### SUMMARY OF THE INVENTION

Therefore, it is the object of the present invention to provide a dose inhalation monitor such that the patient can take a precise dose of medicine upon an appropriate time, thereby leading the patient to an early convalescence.

Another object of the present invention is to provide the dose inhalation monitor having the ability to record automatically the feeding times and the number of feeding operation such that the medical practitioner can make the correct diagnose based on the record.

According to the present invention, a dose inhalation monitor is provided to include a main body for containing medicine therein, a dose monitoring unit and a transmission device. The main body has a feeding mouth and a bypass communicated spatially with the feeding mouth. The dose monitoring unit is disposed on the main body, and includes a detector capable of detecting and converting a pressure within the bypass into a voltage level and a processor capable of processing the voltage level in order to generate an oral dose of medicine corresponding to the voltage level. The transmission device is connected to the dose monitoring unit for transmitting the oral dose of medicine to a distal receiver after receipt thereof.

### BRIEF DESCRIPTION OF THE DRAWINGS

Other features and advantages of this invention will become more apparent in the following detailed description of the preferred embodiment of this invention, with reference to the accompanying drawings, in which:

FIG. 1 is a schematic view of a conventional medicine feeding apparatus;

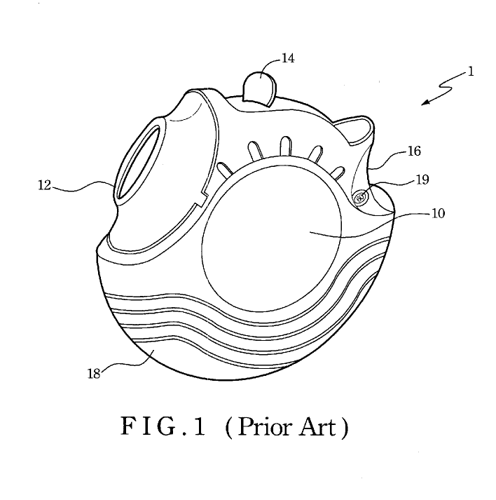

FIG. 2 is a schematic view of a dose inhalation monitor of the present invention;

FIG. 3 is a sectional view of the dose inhalation monitor of the present invention;

FIG. 4 is a block diagram representing a dose monitoring unit employed in the dose inhalation monitor of the present invention; and

FIG. 5 is a circuit diagram representing a detector of the dose monitoring unit employed in the dose inhalation monitor of the present invention.

### DETAILED DESCCRIPTIONS OF THE PREFERRED EMBODIMENT

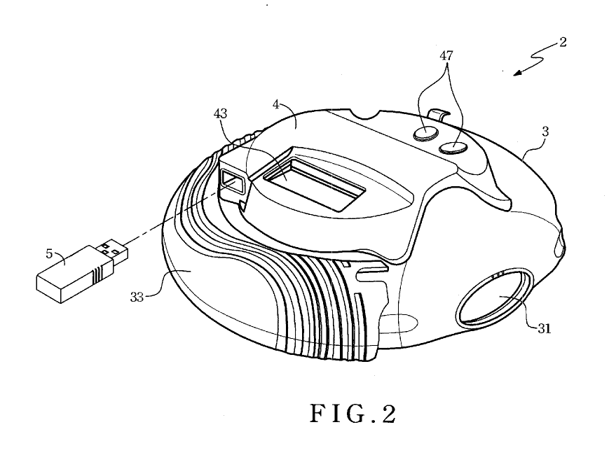

Referring to FIG. 2, a dose inhalation monitor 2 of the present invention is shown to include a main body 3, a dose monitoring unit 4 and a transmission device 5.

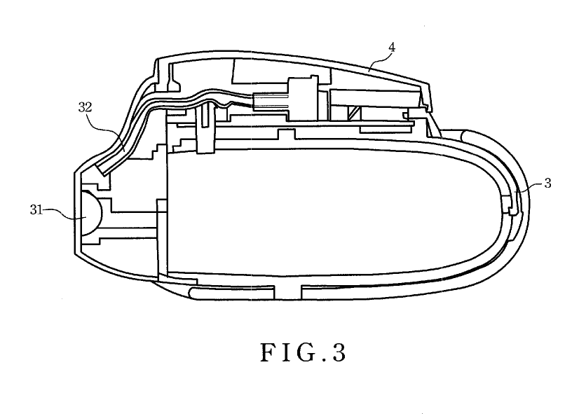

As illustrated in FIGS. 2 and 3, the main body 3 is oval-shaped, and has a lateral side formed with a feeding mouth 31 and a bypass 32, a dispensing arm (not visible) and a slidable shield 33. Since the structure of the main body 3 is similar to that of the conventional ones, a detailed description thereof is omitted herein for the sake of brevity. The only difference resides in that the bypass 32 is communicated spatially with the feeding mouth 31 such that suction of the dose of medicine via the feeding mouth 31 by the patient results in variation of the pressure within the bypass 32.

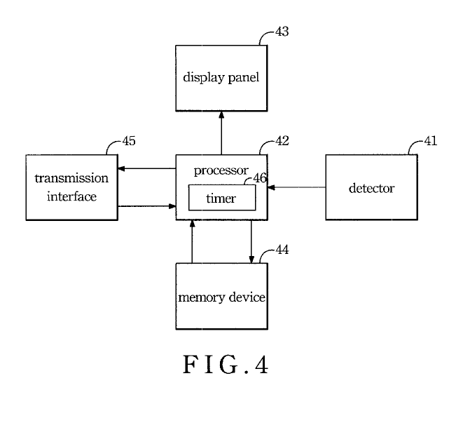

The dose monitoring unit 4 is disposed on the main body 3 in several ways, such as fixedly, detachably or integrally formed with. FIG. 4 illustrates the block diagram representing the dose monitoring unit 4 employed in the dose inhalation monitor 1 of the present invention. The dose monitoring unit 4 includes a detector 41, a processor 42, a display panel 43, a memory device 44 and a transmission interface 45. The detector 41 is capable of detecting a pressure within the bypass 32 and converting the pressure into a voltage level and transmitting the same to the processor 42. The processor 42 is capable of processing the voltage level in order to generate an oral dose of medicine corresponding to the voltage level. In addition, the processor 42 is further capable of determining whether the oral dose of medicine is sufficient and later records the data concerning the oral dose and the voltage level within the memory device 44, which is coupled to the processor 42. The main body 3 further has a dispensing arm (not visible), activation of the dispensing arm results in dispensation of the oral dose of medicine and simultaneously enables the detector 41 to detect a taking procedure of the oral dose of medicine.

The dose monitoring unit 4 further includes a timer 46 installed within the processor 42 such that each time the dose of medicine is dispensed from the bypass 32, the timer 46 will count the dispensing number of the oral dose of medicine and the dispensing time. The display panel 43 is coupled to the processor 42 for displaying the dispensing number of the oral dose of medicine and the dispensing time.

The transmission interface 45 can be a USB connector (or interface), a wireless transmission module (or interface), a Bluetooth module (or interface) and RS 232 connector. The transmission device 5 is connected to the dose monitoring unit 4 in several ways. Preferably, the transmission device 5 is compatible to the transmission interface 45 and is connected detachably to the transmission interface 45 for transmitting the oral dose of medicine to a distal receiver after receipt thereof. The medical practitioner at the distal receiver can diagnose the patient condition based on the received data with the assistance of a computer system.

Referring again to FIG. 2, a disc battery (or button cell battery) serves the power source in the dose monitoring unit 4. The battery strength is displayed in the display panel 43. The dose monitoring unit 4 may generate an alarm in case charging of the battery is due or when replacement is due. The dose monitoring unit 4 may have several function keys 47 on an outer surface thereof. Activation of one specific function key 47 may result in display of the prior data including the following feeding time/patient ID code/battery capacity status/dose times or a cancelled list with the feeding times.

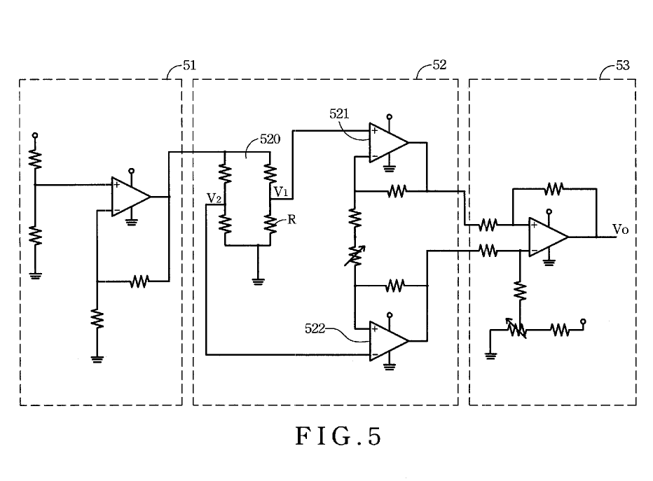

FIG. 5 is a circuit diagram representing the detector of the dose monitoring unit 4 employed in the dose inhalation monitor of the present invention. The circuit diagram includes a stable circuit 51, a detection circuit 52 and an amplifying circuit 53. Because the change amount of pressure within the bypass 32 is relatively small, the stable circuit 51 is utilized in the dose monitoring unit 4 for providing a stable voltage level. The detection circuit 52 includes a bridge circuit 520 and a first amplifier 521 and a second amplifier 522. The bridge circuit 520 has a resistance R, which varies according to the change of the pressure within the bypass 32. Upon variation of the resistance R, two output voltage levels (V1, V2) of the bridge circuit 520 will also alter. At this time, the two output voltage levels are transmitted respectively to the first and second amplifiers 521, 522 for undergoing a prosecution. The result is conveyed to the amplifying circuit 53 for another prosecution in order to achieve the final output voltage level Vo. Afterward, the processor 42 processes algorithm base on the final output voltage level Vo and timing and converts the same into the oral dose of medicine corresponding to the final output voltage level Vo.

The advantages provided by the dose inhalation monitor of the present invention are as follows:
(1) The dose inhalation monitor is capable of determining sufficiency in the oral dose of medicine. Since the dose amount is displayed in the display panel so that the actual situation of the patient is under the observation of the medical personnel.
(2) Since the dose inhalation monitor of the present invention can automatically record the dose, medicine feeding time and the feeding intervals digitally, thereby providing an excellent treatment to the patient.
(3) The recorded data of the patient are transmitted to the distal receiver (medical establishment), the medical practitioner at the distal receiver can make a correct diagnose or analysis of the patient's condition.

While the present invention has been described in connection with what is considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A dose inhalation monitor comprising:
a main body for containing medicine therein, and having a feeding mouth and a bypass communicated spatially with said feeding mouth;
a dose monitoring unit disposed on said main body, and including a detector capable of detecting and converting a pressure within said bypass into a voltage level and a processor capable of processing said voltage level in order to generate an oral dose of medicine corresponding to said voltage level; and
a transmission device connected to said dose monitoring unit for transmitting said oral dose of medicine to a distal receiver after receipt thereof.

2. The dose inhalation monitor according to claim 1, wherein said main body is oval-shaped, and has a lateral side formed with said feeding mouth and a slidable shield provided slidably on said lateral side for covering said feeding mouth.

3. The dose inhalation monitor according to claim 2, wherein said main body further has a dispensing arm, activation of said dispensing arm resulting in dispensation of said oral dose of medicine and simultaneously enabling said detector to detect a taking procedure of said oral dose of medicine.

4. The dose inhalation monitor according to claim 1, wherein said dose monitoring unit further includes a display panel coupled to said processor for displaying said oral dose of medicine.

5. The dose inhalation monitor according to claim 1, wherein said dose monitoring unit further includes a memory device coupled to said processor for recording said oral dose of medicine.

6. The dose inhalation monitor according to claim 1, wherein said dose monitoring unit further includes a timer for counting dispensing number of said oral dose of medicine.

7. The dose inhalation monitor according to claim 1, wherein said transmission device is a wireless transmission module.

8. The dose inhalation monitor according to claim 1, wherein said transmission device is a Bluetooth module.
